(19) United States
(12) Patent Application Publication
(10) Pub. No.: US 2013/0216493 A1
(43) Pub. Date: Aug. 22, 2013

(54) PEG OR PEG BLOCK COPOLYMERS FOR TREATING COLORECTAL CANCER

(71) Applicants: Peter Stein, London (GB); Leighton Jones, Mid-Glamorgan (GB); Samuel Smith, Mid-Glamorgan (GB); Ian Cox, Mid-Glamorgan (GB); Jörg Plessl, Kirchhain (DE); Corinne De Vries, Bath (GB); Rachel Charlton, Bath (GB)

(72) Inventors: Peter Stein, London (GB); Leighton Jones, Mid-Glamorgan (GB); Samuel Smith, Mid-Glamorgan (GB); Ian Cox, Mid-Glamorgan (GB); Jörg Plessl, Kirchhain (DE); Corinne De Vries, Bath (GB); Rachel Charlton, Bath (GB)

(73) Assignee: Norgine BV, Amsterdam Zuid-Oost (NL)

(21) Appl. No.: 13/883,262

(22) PCT Filed: Nov. 4, 2011
(86) PCT No.: PCT/GB11/01561

§ 371 Date: May 2, 2013

Related U.S. Application Data
(60) Provisional application No. 61/412,128, filed on Nov. 10, 2010.

(30) Foreign Application Priority Data
Nov. 4, 2010 (GB) 1018650.0

(51) Int. Cl. A61K 31/765

(52) U.S. Cl. CPC A61K 31/765
USPC 424/78.01

(57) ABSTRACT

The present invention relates to methods for and of treating, ameliorating or preventing colorectal cancer (CRC) in humans using polyethylene glycol (PEG) or a PEG block-copolymer such as Pluronic® F68. Compositions for use in treating, ameliorating and/or preventing CRC comprising PEG are also disclosed. Such compositions may be used in the methods of the invention.

The present invention concerns methods of and for the treatment, amelioration and/or prevention of colorectal cancer (CRC) in humans. The present invention also concerns compositions for use in such methods.

Colorectal cancer is a major cause of death in the human population, particularly in North America and Europe. Prevention is urgently required and therefore research into various strategies including control of diet and other lifestyle modifications, including chemical interventions, has been reported.

In International Patent Application No: WO00/24407, the use of a non-fermented osmotic laxative such as polyethylene glycol (PEG) to treat and prevent colorectal cancer is disclosed. This finding was based on work carried out in an azoxymethane (AOM) model of colon cancer in Fischer F344 rats. It suggests that the doses used in rats may be translated into a daily dose in humans of between 10 to 80 g. However, no direct evidence is disclosed that a therapeutic effect for PEG exists in human forms of CRC nor over what timeframe such an exposure is necessary to achieve such an effect.

Dorval E. et al; Gastro Clin Biol, 30:1196-1199 (2006), investigated, in a human population based study, the prevalence of colorectal adenomas in association with dietary PEG consumption. Consecutive patients, attending hospital for routine total colonoscopy, were asked if they had previously taken a laxative or a non-steroidal anti-inflammatory drug (NSAID). Based on the answers provided, the authors concluded that use of Forlax®, a PEG containing bowel preparation, was associated with a lower incidence of colorectal tumours, yet other PEG or PEG-like laxatives including MOVICOL®, Transipeg® and Idrocol® did not produce significant results. The authors posited that one reason may have been that the “PEG dose varies from one brand to another, and appears to be among the highest in Forlax®. As noted in the paper, since the study was based on a patient based questionnaire, “this survey did not yield reliable information regarding the duration, quantity, regularity or timing of PEG ingestion.”

In addition to the above-noted references, there have been other studies involving rodents building on the work mentioned above, which generally tend to the view that a relatively high dose of a PEG in a relatively short time frame can reduce incidences of ACF and EGFR—believed to be markers for colon cancer. There is no apparent consensus regarding the timing of the administration of the PEG in order to be effective in reduction of the surrogate marker(s) and the level of dose of the PEG, if scaled up for human use, would result in diarrhoea. The requirement for a high dose is echoed in the Dorval (2006) reference, which shows, at table II, that MOVICOL® (a PEG plus electrolytes composition) is ineffective; similarly, it shows that the combined results of PEG-containing laxatives are not significant. To date, this appears to be the only published study related to the human use of PEG in the context of colorectal cancer, yet it does not exclude data from patients whose constipation (and the reason for them taking a laxative such as Forlax® or MOVICOL®) is caused by, or is a symptom of, colorectal cancer.

Surprisingly, when the present inventors instigated a study of a General Practice Research Database (GPRD) to describe the incidences of colorectal cancer in the human population following different types of laxative exposure by comparison with controls sampled from the same laxative cohort population but having no diagnosis of colorectal cancer, they found a potential dose-response relationship in patients ingesting over 60 sachets of MOVICOL®, both 24 and 36 months prior to the earliest date they presented with evidence of a CRC diagnosis. MOVICOL is provided in a sachet containing 13.8 g powder for making up into an oral solution. Each sachet contains: 13.1250 g Macrogol (polyethylene glycol (PEG)) 3350, 0.3507 g sodium chloride, 0.1785 g sodium bicarbonate and 0.0466 g potassium chloride. This is the standard dose of MOVICOL. It also contains flavouring and sweetener. MOVICOL has been on the market since 1995.

Therefore, in accordance with the present invention there is provided a composition for use in a method for treating, ameliorating and/or preventing CRC in a human comprising administering to the human approximately 800 grams or greater (e.g. 2362 grams or greater) of PEG or PEG block co-polymer over a period of 36 consecutive calendar months.

In accordance with the present invention there is provided a composition for use in a method for treating, ameliorating and/or preventing CRC in a human comprising administering to the human approximately 800 grams or greater (e.g. 2362 grams or greater) of PEG or PEG block co-polymer over a period of 24 consecutive calendar months.

In accordance with the present invention there is provided a composition for use in a method for treating, ameliorating and/or preventing CRC in a human comprising administering to the human an effective and sub-laxative amount of a PEG or PEG block co-polymer.

In one aspect of the invention the composition of the invention is for use in treating CRC in a human. In another aspect, the composition of the invention is for ameliorating CRC in a human. In a further aspect, the composition of the invention is for use in preventing CRC in a human.

In another aspect of the invention, there is provided a method of treating, ameliorating and/or preventing colorectal cancer (CRC) in a human which method comprises administering to the human an effective and sub-laxative dose of PEG or PEG block co-polymer.

In another aspect of the invention there is provided a method for treating, ameliorating and/or preventing CRC in a human which method comprises administering to the human an effective amount and sub-laxative dose of PEG or PEG block co-polymer.

In another aspect of the invention there is provided a method; (a) for preventing constipation and; (b) of preventing CRC in a human which method comprises administering an effective and sub-laxative dose of PEG or PEG block co-polymer.

In another aspect of the invention there is provided a method; (a) for preventing constipation and; (b) for preventing CRC in a human which method comprises administering an effective and sub-laxative dose of PEG or PEG block co-polymer.

In another aspect of the invention there is provided a method; (a) for preventing constipation and; (b) for ameliorating CRC in a human which method comprises administering to the human an effective and sub-laxative dose of PEG or PEG block co-polymer.

In another aspect of the invention there is provided a method; (a) for preventing constipation and; (b) for treating CRC in a human which method comprises administering to the human an effective and sub-laxative dose of PEG or PEG block co-polymer

In another aspect, there is provided a composition suitable for human administration comprising, as a unit dose, a sub-laxative amount of PEG or PEG block co-polymer.

In another aspect, there is provided a composition for use in a method of the invention described supra comprising, as a unit dose, a sub-laxative amount of PEG or PEG block co-polymer.

The term “sub-laxative amount” means an amount that does not increase the average number of stools per week for the human subject concerned.

The term “sub-laxative dose”, means a dose that does not increase the average number of stools per week for the human subject concerned.

The term “PEG” means polyethylene glycol having the general formula H—(O—CH₂—CH₂)n —OH.

The term “PEG block co-polymer” refers to a co-polymer of polyethyleneglycol with polypropylene glycol, namely, polyethylenepolypropylene glycol. Exemplary PEG block co-polymers include those available under the tradename “Pluronic® F68” or “poloxamer 188”.

The term “treating” and grammatical variations thereof is intended to mean that methods and compositions of the invention may be able to cure and/or reverse the normal disease course of CRC, particularly early stage colorectal pathologies associated with the development of CRC, for example by treating colon polyps.

The term “ameliorating” and grammatical variations thereof is intended to mean that methods and compositions of the invention can slow or stop the progression of early stage colorectal pathologies towards the development of CRC, e.g. slow or stop the further development of colon polyps and/or aberrant crypt foci (ACF) into CRC.

The term “preventing” and grammatical variations thereof is intended to mean that methods and compositions of the invention reduce the risk of developing CRC and/or (as the case may be) constipation.

The term “colorectal cancer” or “CRC” refers to colorectal, colon and/or rectal cancer in humans.

The term “one month” means a contiguous 30 day period.

The phrase “a method for” is intended to denote a purposive method.

In certain embodiments of the invention described supra, a dose (e.g. effective and sub-laxative dose) is administered to provide approximately 800 grams or greater (e.g. 2362 grams or greater) of PEG or PEG block co-polymer over a period of 36 consecutive calendar months. In certain other embodiments of the invention described supra, a dose (e.g. an effective and sub-laxative dose) is administered to provide between 800 and 2365 (e.g. 800.6 to 2362.5) grams of PEG or PEG block co-polymer over a period of 36 consecutive calendar months.

In certain embodiments of the invention described supra, a dose (e.g. effective and sub-laxative dose) is administered to provide approximately 800 grams or greater (e.g. 2362 grams or greater) of PEG or PEG block co-polymer over a period of 24 consecutive calendar months. In certain other embodiments of the invention described supra, a dose (e.g. an effective and sub-laxative dose) is administered to provide between 800 and 2365 (e.g. 800.6 to 2362.5) grams of PEG or PEG block co-polymer over a period of 24 consecutive calendar months.

In other embodiments of the invention described supra, a dose (e.g. an effective and sub-laxative dose) is administered to provide 266 grams or greater (e.g. 400 grams or greater; 787 grams or greater; 1181 grams or greater) of PEG or PEG block co-polymer over a period of 12 consecutive calendar months. In certain embodiments of the invention described supra, a dose (e.g. an effective and sub-laxative dose) is administered to provide between 266 grams and 1181 grams (e.g. between 266.9 grams and 787.5 grams; between 400.3 grams and 1181.3 grams; between 400.3 grams and 787.5 grams) of PEG or PEG block co-polymer over a period of 12 consecutive calendar months.

In other embodiments of the invention described supra, a dose (e.g. an effective and sub-laxative dose) is administered to provide 22 grams or greater (e.g. 33 grams or greater; 65 grams or greater; 98 grams or greater) of PEG or PEG block co-polymer over a period of one month. In certain embodiments of the invention described supra, a dose (e.g. an effective and sub-laxative dose) is administered to provide between 22.2 grams and 98.4 grams (e.g. between 33.3 grams and 98.4 grams; 22.2 grams and 65.6 grams; 65.6 grams and 98.4 grams) of PEG or PEG block co-polymer over a period of one month.

In other embodiments of the invention described supra, a dose (e.g. an effective and sub-laxative dose) is administered to provide 5.1 grams or greater (e.g. 7.7 grams or greater; 15.1 grams or greater; 22.7 grams or greater) of PEG or PEG block co-polymer over a period of one week. In certain embodiments of the invention described supra, a dose (e.g. an effective and sub-laxative dose) is administered to provide between 5.1 grams and 22.7 grams (e.g. between 7.7 grams and 22.7 grams; 5.1 grams and 15.1 grams; 15.1 grams and 22.7 grams) of PEG or PEG block co-polymer over a period of one week.

In other embodiments of the invention described supra, a dose (e.g. an effective and sub-laxative dose) is administered to provide 0.73 grams or greater (e.g. 1.1 grams or greater; 2.2 grams or greater; 3.2 grams or greater) of PEG or PEG block co-polymer over a period of one day. In certain embodiments of methods of the invention described supra, a dose (e.g. an effective and sub-laxative dose) is administered to provide between 0.73 grams and 3.22 grams (e.g. between 1.1 grams and 3.2 grams; 0.73 grams and 2.2 grams; 2.2 grams to 3.2 grams) of PEG or PEG block co-polymer over a period of one day. Such doses may be administered daily over period of 24 or 36 consecutive calendar months. Alternatively, such doses may be administered intermittently over a period of 24 or 36 consecutive calendar months.

In all embodiments described herein, it will be apparent to the reader of this specification that ranges specified as being “between” or “within” two values are to be understood as being inclusive of those values, for example “between 1.1 grams and 3.2 grams” includes 1.1 grams and 3.2 grams respectively.

In other embodiments of the invention, compositions for use in a method (such as the methods described supra) for treating, ameliorating and/or preventing CRC in humans comprising an amount (e.g. effective and sub-laxative) of a PEG or PEG block co-polymer are provided.

In certain embodiments, the composition of the invention is in the form of a tablet (e.g. chewable or suckable tablet), capsule, caplet, troche, liquid, powder (e.g. powder for solution or suspension) and granules. Preferably, it is the form of a solid composition for oral administration. It may be a solid tablet, for example a chewable and/or suckable tablet.

In some embodiments of the invention, the PEG has an average molecular weight of at least 400, or at least 1000, or between 2000 to 10000 Daltons (e.g. 2500 to 8500, preferably 3000 to 8000 (e.g. 6000 or 8000), more preferably 2500 to 4500, e.g. 3350 or 4000). In certain embodiments, a blend of PEG molecules is provided of differing molecular weights to give a desired overall average molecular weight (for example an average molecular weight of 3350 or thereabout, or an average molecular weight of 4000 or thereabout). Exemplary PEG products are macrogol 3350, macrogol 4000, macrogol 6000, macrogol 8000 and are available commercially.

In some embodiments, the composition of the invention is for treating CRC. In some embodiments, the composition of the invention is for ameliorating CRC. In some embodiments, the composition of the invention is for preventing CRC.

Accordingly, the present invention provides a solid composition for oral administration as a solid for treating, ameliorating and/or preventing CRC comprising:
(a) 50-90% w/w polyethylene glycol (PEG) having an average molecular weight within the range 2,000 to 10,000 Da; and
(b) 10-40% w/w of a solid (sometimes referred to herein as “solid of component (b)”)

The invention provides a solid composition for oral administration as a solid for treating, ameliorating and/or preventing CRC comprising:
(a) 50-90% w/w polyethylene glycol (PEG) having an average molecular weight within the range 2,000 to 10,000 Da;
(b) 10-40% w/w of a solid; and optionally
(c) q.s. to 100% w/w of further excipients such as flavourings, sweeteners and lubricants.

Herein, “% w/w” of a component is understood to mean the proportion, as a percentage, that the weight of the respective component makes up of the total weight of the solid composition.

The present invention further provides a solid composition for oral administration as a solid for treating, ameliorating and/or preventing CRC comprising:
(a) 50-90% w/w polyethylene glycol (PEG) having an average molecular weight within the range 2,000 to 10,000 Da; and
(b) 1040% w/w of a solid selected from the group; sorbitol, lactose, dextrates, cellulose, xylitol, maltitol, mannitol.

The present invention further provides a solid composition for oral administration as a solid for treating, ameliorating and/or preventing CRC comprising:
(a) 50-90% w/w polyethylene glycol (PEG) having an average molecular weight within the range 2,000 to 10,000 Da; and
(b) 10-40% w/w of a solid selected from the group: sorbitol, lactose, lactose and starch (e.g. a compound comprising lactose monohydrate and maize starch such as Starlac®), dextrates, cellulose (such as microcrystalline cellulose), xylitol, maltitol and mannitol.

Preferably, the solid of component (b) is greater than 10% w/w, preferably greater than 12% w/w, more preferably greater than 15% (e.g. 15% to 17%) of the solid composition. Preferably the weight ratio of component (a) to component (b) is 1.25:1 to 9:1, preferably 2:1 to 7:1, preferably 4:1 to 6:1. In preferred embodiments, the ratio of component (a) to component (b) is approximately 5:1.

Preferably, the solid composition is chewable and/or suckable. It may be a solid tablet, for example a chewable and/or suckable tablet.

It has surprisingly been found that a solid composition of the invention comprising PEG component (a) and solid component (b) is pleasantly chewable or suckable, has good taste, structural integrity and beneficial manufacturing properties. By “chewable” or “suckable” is meant herein that the solid composition is for oral administration and is capable of being chewed or sucked in the mouth so that the first step in the digestive process starts in the buccal cavity.

Compositions of the invention may further comprise electrolytes. Compositions of the invention are preferably substantially free from electrolytes.

For example, they are preferably substantially free from sodium chloride, potassium chloride and sodium bicarbonate. They are preferably substantially free from sulphates or phosphates, for example, they are particularly preferred to be substantially free from sodium sulphate. Compositions of the invention are preferably substantially free from carbonates, bicarbonates, alkali metal ions and halide ions. Compositions of the present invention are most preferably substantially free from sodium, potassium, chloride, bicarbonate, carbonate and/or sulphate ions. In many instances, flavourings, lubricants and sweeteners may contain small amounts of electrolytes. Such amounts are not considered herein to be “substantial”. Compositions of the invention are preferably substantially free from alginates and/or ascorbates and/or citrates. By “substantially free from” herein is also meant that the ingredient is not added to the composition during preparation or manufacture.

In some embodiments, compositions of the present invention are substantially free from any osmotic agent other than PEG or PEG block co-polymer.

The polyethylene glycol (PEG) for use in a solid composition of thoinvention preferably has an average molecular weight (for example a weight average molecular weight), in Daltons, within the range 2,000 to 10,000, preferably 2,500 to 8,500, preferably 3,000 to 8,000, more preferably 3,000 to 6,000, more preferably 2,500 to 6,500, more preferably 2,500 to 4,500 for example 3,000 to 4,500, for example 3,000 to 4,100, for example 3,000 to 4,000. The PEG may have an average molecular weight within the range 6,000 to 10,000, for example 7,000 to 9,000. For example, the PEG may be, or comprise PEG 3,350, PEG 4,000 or PEG 8,000 as defined in national or regional pharmacopoeias. Further examples of suitable PEGs recognized in some national pharmacopoeias include Macrogols, for example Macrogol 4,000. Optionally, the PEG used in compositions of the invention may comprise two or more different PEG components. Optionally, the PEG used in compositions may have at least two differing average molecular weights. PEG of the relevant molecular weights in a form suitable for use in humans is available commercially.

In a preferred embodiment, PEG is present in the solid composition in an amount of 60 to 90% w/w, preferably 70 to 90% w/w, more preferably 70 to 89% w/w, for example 75 to 89% w/w. In a further embodiment, PEG is present in an amount of 78 to 89% w/w, for example 80 to 85% w/w, for example 81 to 85% w/w, for example 80 to 84% w/w, for example 82 to 84% w/w. In a further embodiment, PEG is present in an amount of 50 to 80% w/w, for example 60 to 80% w/w, for example 70 to 80% w/w, for example 70 to 79% w/w, for example 75 to 79% w/w.

In some embodiments, the composition comprises between 0.1 to 6.5 grams (e.g. 0.73 grams to 3.2 grams; 1.1 grams to 3.2 grams; 0.73 grams to 2.2 grams) of PEG or PEG block co-polymer. In other embodiments, the composition comprises 5 grams or less of PEG or PEG block co-polymer. In other embodiments the composition comprises between 0.2 grams to 6.5 grams (e.g. 0.3 grams to 5.0 grams) of PEG or PEG block co-polymer. In further embodiments, the composition comprises between 1 to 4 grams of PEG or PEG block co-polymer. In other embodiments, the composition comprises between 1.5 and 2.5 grams of PEG or PEG block co-polymer. In other embodiments, the composition comprises between 2.0 grams to 2.5 grams of PEG or PEG block co polymer, for example 2.2 grams or thereabout.

The solid of component (b) is preferably selected from the group consisting of sorbitol, lactose, lactose and starch, dextrates, cellulose (e.g. microcrystalline cellulose), xylitol, maltitol, mannitol. Lactose or similar ingredients may be present in hydrated form.

Where the solid of component (b) is lactose and starch, the lactose component maybe in the form of a monohydrate. The starch component maybe derived from any suitable source such as wheat starch, maize starch, potato starch and rice starch. The lactose component may make up 50% to 95% of the lactose/starch solid, for example 60% to 90%, e.g. 70 to 85% such as 85%.

Solids of component (b) are preferably of a purity and grade suitable for consumption by e.g. humans.

The solid of component (b) makes up 10 to 40% w/w of the solid composition. In a preferred embodiment, the solid of component (b) makes up 10 to 30% w/w of the solid composition, preferably greater than 10% w/w up to (and including) 30% w/w. For example, the solid of component (b) makes up 10 to 25% w/w, for example 10 to 20% w/w, preferably 12 to 20% w/w, more preferably 12 to 19% w/w, 12 to 18% w/w, or 12 to 17% w/w. For example, the solid of component (b) may make up 14 to 20% w/w, 14 to 19% w/w or 14 to 18% w/w 14 to 17% w/w of the solid composition such as 15 to 16.5% w/w of the solid composition of the invention.

Preferably, the solid of component (b) is mannitol. It has been found that a solid composition comprising PEG and mannitol is more palatable than a solid composition comprising PEG and no mannitol, even if flavouring is added. In particular, it has been found that a tablet comprising PEG and mannitol has a much lower requirement for lubricant or lubrication during tablet manufacture than a tablet comprising PEG but no mannitol. A high level of a lubricant in a tablet generally makes the tablet have an unacceptable taste. The reduced level (or absence of) a lubricant as compared with a tablet comprising PEG but no mannitol brings about an improved palatability (taste and mouthfeel) of a chewable or suckable tablet comprising PEG and mannitol. Such tablets are thus particularly suitable for use in the current invention.

Typically, solid compositions of dry ingredients are manufactured using dry granulation followed by punching with punch and die equipment. In a punch and die machine, dry ingredients are compressed together. It has surprisingly been found that a solid composition comprising PEG and mannitol in the specified proportions has better structural integrity and is more convenient to manufacture than a solid composition comprising PEG and no mannitol, or a smaller proportion of mannitol. Solid compositions of that type are less susceptible to capping and laminating during punch and die manufacture than solid compositions comprising a smaller proportion of mannitol, or no mannitol. Solid compositions that become capped or laminated during die pressing are not suitable for use and they become waste. It has been found that a solid composition containing between 50 and 90% w/w PEG and 10 to 40% w/w mannitol has better tablet pressing characteristics than a solid composition containing no mannitol or 10% w/w or less mannitol, for example less than 10% mannitol. Such compositions are thus particularly suitable for use in the current invention.

Mannitol may make up 10 to 40% w/w of the solid composition. In a preferred embodiment, mannitol makes up 10 to 30% w/w of the solid composition. For example, mannitol makes up 10 to 25% w/w, for example 10 to 20% w/w, preferably 12 to 20% w/w, more preferably 12 to 19% w/w, 12 to 18% w/w, or 12 to 17% w/w. For example, mannitol may make up 14 to 20% w/w, 14 to 19% w/w or 14 to 18% w/w 14 to 17% w/w of the solid composition of the invention. Mannitol may be provided in various physical forms. For example, mannitol is available commercially in granular, powder or spray-dried form. In a preferred embodiment, the mannitol is granular. Mannitol is commercially available from several suppliers, including Merck, SPI Polyols Inc and Roquette.

In an embodiment, the PEG and mannitol are present in a weight ratio of PEG:mannitol of 1.25:1 to 9:1 (e.g. 3:1 to 9:1, or 4:1 to 9:1), preferably 2:1 to 7:1, preferably 4:1 to 6:1 or 4:1 to 8:1, for example 5:1 to 6:1. In preferred embodiments the ratio of PEG to mannitol 5:1 or thereabout.

The structural integrity of the solid composition is retained when the mannitol is granular mannitol. It is surprising that the solid composition of the invention is so structurally sound with granular mannitol. In general it is found that granular mannitol cannot be used with concentrations of other materials exceeding 25% by weight (Handbook of Pharmaceutical Excipients, 5^{th} Ed., Pharmaceutical Press, 2006, page 452). It has been found that solid compositions for use in the invention, which comprise 60 to 90% w/w of materials other than mannitol, are readily manufacturable and have good structural integrity.

It has also surprisingly been found that a solid composition for use in the invention, comprising PEG and mannitol in the specified proportions is less prone to sticking to punch and die equipment than a solid composition comprising PEG and a smaller proportion of mannitol, or no mannitol. This is particularly important when manufacturing compositions of the present invention at commercial scale since fouling of the manufacturing machinery may lead to manufacturing down-time with the increased costs associated therewith.

Lubricants can be included in tablet compositions to reduce the propensity for them to stick to the punch or die after die pressing. Examples of lubricants include magnesium stearate, potassium stearate, talc, stearic acid, sodium lauryl sulphate, and paraffin. Mixtures of different lubricants may be used. It has been found that a solid composition for use in the invention, comprising PEG and mannitol in the specified proportions, requires a smaller proportion of lubricant to satisfactorily avoid sticking than a tablet comprising PEG and a smaller proportion of mannitol, or no mannitol. Preferably, a solid composition comprises lubricant in an amount of 2.0% w/w or less, for example 1.5% w/w or less, or 1.0% w/w or less. For example it may comprise lubricant in an amount of 0.1 to 0.9% w/w, for example 0.2 to 0.8% w/w, preferably 0.3 to 0.7% w/w. For example, the lubricant is present in ratio of solid of component (b) (such as mannitol): lubricant ratio of 170:1 to 16:1, for example 57:1 to 20:1. A particularly preferred lubricant is magnesium stearate. If the lubricant is magnesium stearate, it is effective to satisfactorily avoid sticking when used at a level of under 1% w/w. Accordingly, in an embodiment, the tablet of the invention further comprises magnesium stearate in an amount of 0.1 to 0.9% w/w, for example 0.2 to 0.8% w/w, preferably 0.3 to 0.7% w/w, more preferably 0.5% w/w. It is surprising that magnesium stearate is effective at these levels as, in general, magnesium stearate is required at a level of over 1% in compositions comprising mannitol (Handbook of Pharmaceutical Excipients, 5^{th} Ed., Pharmaceutical Press, 2006, page 452).

Thus the present invention provides a solid composition for administration as a solid for treating, ameliorating and/or preventing CRC comprising;
(a) 50-90% w/w polyethylene glycol (PEG) having an average molecular weight within the range 2,000 to 10,000 Da;
(b) 10-40% w/w of mannitol; and
(c) 0.1 to 0.9% (e.g. 0.2 to 0.8%, 0.3 to 0.7% such as 0.5%) w/w of a lubricant such as magnesium stearate.

In some embodiments, the ratio of mannitol:lubricant is preferably 10:1 or greater, preferably, 20:1 or greater e.g. 25:1 or greater such as 30:1 or greater (e.g. 30:1 to 35:1 such as 30.6:1 or 32.4:1).

In an embodiment, a solid composition of the invention does not include any added flavouring. In a preferred embodiment, a solid composition of the invention includes at least one flavouring. Suitable flavourings are available from various flavour manufacturers and suppliers, for example International Flavours and Fragrances Inc. (Duddery Hill, Haverhill, Suffolk, CB9 8LG, United Kingdom), Ungerer & Company (Sealand Road, Chester, CH1 4LP, United Kingdom), Firmenich (Firmenich UK Ltd., Hayes Road, Southall, Middlesex, UB2 SNN, United Kingdom) or S. Black Ltd (Foxholes Business Park, John Tate Road, Hertford, Herts, SG13 7YH, United Kingdom). Examples of suitable flavours include orange, lemon-lime, lemon, citrus, chocolate, tropical fruit, aloe vera, peppermint, tea, strawberry, grapefruit, blackcurrant, pineapple and vanilla, raspberry-lemon, cola flavour, and combinations thereof.

Preferred flavours are peppermint and raspberry-lemon flavour.

A flavouring may be integral in a solid composition, or it may be coated onto its surface. In one embodiment, the flavouring is integral in the solid composition. In such a solid composition, the flavouring preferably makes up 0.1 to 15% w/w of the solid composition. For example, the flavouring may make up 0.1 to 5% w/w of the solid composition, for example 0.1 to 2.0% w/w, for example 0.2 to 2.0% w/w. When the flavouring is peppermint, it is preferably present at a level of 0.1 to 1.0% w/w, for example 0.15 to 0.5% w/w. This level is particularly preferred when the solid of component (b) such as mannitol is present at a level of 14 to 17% w/w of the solid composition of the invention. When the flavouring is raspberry-lemon, it is preferably present at a level of 0.5 to 2.0% w/w, for example 1.0 to 2.0%, for example 1.2 to 1.8% w/w. This level is particularly preferred when the solid of component (b) such as mannitol is present at a level of 14 to 17% w/w of the solid composition of the invention.

In an embodiment, the solid of component (b) such as mannitol and flavouring are, for example, present in a ratio of solid:flavouring of 170:1 to 3:1; when the flavouring is peppermint, the solid of component (b) such as mannitol and flavouring are preferably present in a ratio of solid:flavouring of 113:1 to 28:1. When the flavouring is raspberry-lemon, the solid of component (b) such as mannitol and flavouring are preferably present in a ratio of solid of component (b): flavouring of 14:1 to 7:1.

A solid composition of the invention may comprise one or more sweeteners. Sweeteners may be sugar-based. Preferably, they are not sugar-based. Preferred sweeteners include aspartame, acesulfame potassium (acesulfame K), sucralose and saccharine or combinations thereof. Alternatively, it can be preferred for compositions of the invention to be substantially free from added sweeteners, for example to minimize the number of different components in the compositions. When present, sweeteners may, for example, be present in an amount of 0.01 to 1 w/w. More preferably, a sweetener may be present in an amount of 0.1 to 1% w/w. The level of sweetener required to obtain a satisfactory taste may depend on the presence, and identity and quantity, of the other components of the composition.

In general it is not necessary for a solid composition of the invention to include preservatives or anti-oxidants. Nevertheless, low levels of anti-oxidants or preservatives may be included if required. It is preferred that compositions of the present invention are also substantially free from “salt taste” masking agents, such as agents that mask the taste of sodium sulphate, (other than flavourings mentioned herein) and from salts of non-fatty acids such as salts of mineral acids.

A solid composition of the invention can be of any convenient size. As mentioned above, a tablet should be sufficiently large to provide the desired quantity of PEG to the subject, but not be so large as to be uncomfortable in the mouth, difficult to chew or suck, or difficult to package. A tablet may, for example, have a mass of 0.5 to 10 g, more preferably 0.5 to 5 g, for example 1.0 to 5.0 g, for example 2.0 to 3.5 g, for example 2.5 to 3.5 g. In one embodiment, a tablet of the invention has a mass of from 2.5 to 3.0 g, for example 2.75 g. For certain uses, where a larger amount of PEG is to be delivered to the subject, a larger tablet may be convenient, for example having a mass of 3 to 10 g, for example 3 to 5 g, 3 to 7 g, 4 to 7 g, or 5 to 8 g, for example 4 to 7 g. For certain uses, where a smaller amount of PEG is to be delivered to the subject, for example for paediatric uses, a smaller tablet may be convenient, for example having a mass of 0.5 to 2.0 g, for example 1.0 to 1.75 g, for example 1.25 to 1.50 g.

A solid composition of the invention for treating, ameliorating and/or preventing CRC may therefore be a solid composition of mass 2.0 to 3.5 g comprising:
(a) 1.00-3.15 g polyethylene glycol (PEG) having an average molecular weight within the range 2,000 to 10,000 Da; and
(b) 0.20-1.40 g of solid such as mannitol.

A solid composition of the invention for treating, ameliorating and/or preventing CRC may therefore be a solid composition of mass 2.5 to 3.5 g comprising:
(a) 1.25-3.15 g polyethylene glycol (PEG) having an average molecular weight within the range 2,000 to 10,000 Da; and
(b) 0.25-1.40 g of solid such as mannitol.

Similarly, a composition of the invention for treating, ameliorating and/or preventing CRC may therefore be a solid composition of mass 1.0 to 1.75 g comprising:
(a) 0.50-1.575 g polyethylene glycol (PEG) having an average molecular weight within the range 2,000 to 10,000 Da; and
(b) 0.10-0.70 g of solid such as mannitol.

As mentioned above, a lubricant (for example magnesium stearate) may be present in a solid composition of the invention in an amount of 2% w/w or less, for example 1% w/w or less. A solid composition of the invention of mass 2.0 to 3.5 g may therefore comprise 0.07 g or less of lubricant, for example 0.35 g or less of lubricant. For example, it may comprise lubricant in an amount of 0.002 to 0.0315 g, for example 0.004 to 0.028 g, for example 0.006 to 0.0245 g. A larger composition of the invention of mass 3.0 to 7.0 g may comprise 0.14 g or less of lubricant, for example 0.07 g or less of lubricant. For example, it may comprise lubricant in an amount of 0.003 to 0.063 g, for example 0.006 to 0.056 g, for example 0.009 to 0.049 g. A smaller composition of the invention of mass 1.0 to 1.75 g may comprise 0.035 g or less of lubricant, for example 0.0175 g or less of lubricant. For example, it may comprise lubricant in an amount of 0.001 to 0.01575 g, for example 0.002 to 0.014 g, for example 0.003 to 0.01225 g.

As mentioned above, flavouring may be present in a solid composition of the invention and, when present, it preferably makes up 0.1 to 15% w/w of the solid composition. A solid composition of the invention of mass 2.0 to 3.5 g may therefore comprise 0.002 to 0.525 g of flavouring, for example 0.002 to 0.175 g, for example 0.002 to 0.07 g, for example 0.004 to 0.07 g of flavouring. A larger composition of the invention of mass 3.0 to 7.0 g may comprise 0.003 to 1.05 g of flavouring, for example 0.003 to 0.35 g, for example 0.003 to 0.14 g, for example 0.006 to 0.14 g of flavouring. A smaller composition of the invention of mass 1.0 to 1.75 g may comprise 0.001 to 0.2625 g of flavouring, for example 0.001 to 0.0525 g for example 0.001 to 0.021 g, for example 0.002 to 0.021 g of flavouring.

For example, a solid composition of the invention for treating, ameliorating and/or preventing CRC may comprise:
(a) 50-90% w/w polyethylene glycol (PEG) having an average molecular weight within the range 2,000 to 10,000 Da;
(b) 10-30% w/w of solid such as mannitol;
(c) 0.1-2.0% w/w lubricant; and
(d) 0.1-15% w/w flavouring.

In one embodiment, a solid composition of the invention for treating, ameliorating and/or preventing CRC comprises:
(a) 70-90% w/w polyethylene glycol (PEG) having an average molecular weight within the range 3,000 to 8,000 Da;
(b) 10-25% w/w of solid such as mannitol;
(c) 0.1-1.5% w/w magnesium stearate; and
(d) 0.1-2.0% w/w flavouring.

For example, a solid composition of the invention for treating, ameliorating and/or preventing CRC comprises:
(a) 75-89% w/w polyethylene glycol (PEG) having an average molecular weight within the range 3,000 to 4,000 Da;
(b) 10-20% w/w of solid such as mannitol;
(c) 0.2-0.8% w/w magnesium stearate; and
(d) 0.1-1.0% w/w flavouring.

For example, a solid composition of the invention for treating, ameliorating and/or preventing CRC may comprise:
(a) 1.00-3.15 g polyethylene glycol (PEG) having an average molecular weight within the range 2,000 to 10,000 Da;
(b) 0.20-1.40 g of solid such as mannitol;
(c) 0.002-0.07 g lubricant; and
(d) 0.002-0.525 g flavouring.

In one embodiment, a solid composition of the invention for treating, ameliorating and/or preventing CRC comprises:
(a) 1.40-3.15 g polyethylene glycol (PEG) having an average molecular weight within the range 3,000 to 8,000 Da;
(b) 0.20-0.875 g of solid such as mannitol;
(c) 0.002-0.0525 g magnesium stearate; and
(d) 0.002-0.07 g flavouring.

For example, a solid composition of the invention for treating, ameliorating and/or preventing CRC comprises:
(a) 1.5-3.115 g polyethylene glycol (PEG) having an average molecular weight within the range 3,000 to 4,000 Da;
(b) 0.20-0.70 g of solid such as mannitol;
(c) 0.004-0.016 g magnesium stearate; and
(d) 0.002-0.035 g flavouring.

For example, a solid composition of the invention for treating, ameliorating and/or preventing CRC comprises:
(a) 2273 to 2284 mg polyethylene glycol (PEG) having an average molecular weight within the range 3,000 to 4,000 Da;
(b) 420 to 446 mg of solid such as mannitol;
(c) 13.5 to 13.75 mg magnesium stearate; and
(d) 11 to 42 mg of flavouring such as peppermint or raspberry/lemon flavouring.

The solid compositions of the invention may be packaged in any convenient fashion. For example a plurality of units (e.g. tablets) of the solid composition of the present invention (such as 5, 10, 15 or 20) may be packaged in a way conventional in the vitamin supplements industry. For example, they may be packed in a tube (such as a PTFE tube) equipped with a removable and replaceable closing means, for example a stopper. Alternatively, the solid compositions of the invention may be provided in a jar or other container with a removable and replaceable lid, or in a bag or within a wrapper (for example a foil wrapper). A desiccant is preferably also present. Alternatively, they may be packaged in a blister pack. In an embodiment, the solid compositions are packaged within a tube, jar, bag, wrapper or other container without any wrapping around individual units (e.g. tablets). Optionally, individual units of solid compositions of the present invention may have a wrapping. In a preferred embodiment, 30 units of the composition of the present inventions are provided spilt into three tubes (e.g. 10 units per tube) or other packaging optionally together with instructions for use. Units of the present invention may also be provided in a refill bag enabling previously obtained tubes to be refilled with units of the invention.

The solid compositions of the invention can be taken on their own as presented and chewed or sucked by a subject. It is not necessary for a subject to take water or another drink with the solid composition. Some subjects may wish to drink water or another fluid with or soon after taking a solid composition of the invention so as to facilitate the intake. The convenient packaging and the lack of a need to take water or another drink greatly increases the convenience of the solid compositions to subjects in comparison with other forms of PEG-based products currently on the market. Compositions of the present invention may be consumed prior to eating a meal or snack, together with the meal or snack or following a meal or snack.

In one embodiment, the subject typically takes up to 6 g (or thereabout) per day of PEG, for example 2 to 6 g per day, for example 3 to 5 g per day, for example 4 to 5 g per day. In that embodiment, the composition is free from components of a nature and quantity having a laxative effect. PEG is not considered to have significant laxative activity in an adult when taken at a level of 6 g per day. Mannitol, flavouring and lubricant components are also not considered to have significant laxative activity at the daily levels at which they are provided when the composition provides up to 6 g PEG per day. For a solid composition of 2.0 to 3.5 g total mass and comprising 85% PEG w/w, a healthy subject may be recommended to take 1 or 2, or 1, 2 or 3 per day (to provide up to 6 g or thereabout of PEG per day). For a smaller solid composition (of, for example, 1.0 to 1.75 g total mass), a healthy subject may be recommended to take 1 to 6 per day, for example 2 to 5 per day (to provide up to 6 g or thereabout of PEG per day). Conversely, for a larger solid composition (of, for example, total 3.0 to 7.0 g total mass), a healthy subject may be recommended to take 1 or 2 per day (to provide up to 6 g or thereabout of PEG per day).

Thus, the present invention provides a solid composition for oral administration as a solid (preferably having a mass of 1.0 to 5.0 g) to a healthy subject (such as a human) for use in a method for treating, ameliorating and/or preventing CRC and optionally preventing constipation,
wherein the composition comprises:
(a) 50-90% w/w (for example 60 to 90% w/w, preferably 70 to 90% w/w, more preferably 70 to 89% w/w, for example 75 to 89% w/w, e.g. 78 to 89% w/w, 80 to 85% w/w, 81 to 85% w/w, 80 to 84% w/w, 82 to 84% w/w) polyethylene glycol (PEG) having an average molecular weight within the range 2,000 to 10,000 Da; and
(b) 10-40% w/w (for example 10 to 25% w/w, 10 to 20% w/w, preferably 12 to 20% w/w, more preferably 12 to 19% w/w, 12 to 18% w/w, or 12 to 17% w/w, e.g. 14 to 20% w/w, 14 to 19% w/w or 14 to 18% w/w 14 to 17% w/w) of a solid such as mannitol; together with
(c) optional lubricant, optional flavouring and/or optional sweetener as described supra; and
wherein the method comprises administering said composition so that the subject consumes up to 6 g (or thereabout) of PEG per day.

Preferably, said method comprises administering said composition so that the subject consumes between 2 g and 6 g, e.g. 2 g to 5.5 g per day. Preferably, said method is performed on a daily or alternate day basis. Preferably the method is performed over a period of at least two weeks, preferably at least a calendar month, more preferably at least 6 consecutive calendar months, or at least 12 consecutive calendar months, or at least 24 or at least 36 consecutive calendar months.

In certain embodiments of the invention described supra, the human subject may be one with a predisposition towards developing CRC based, e.g. on family history (e.g. family history of developing CRC), medical history (e.g. treatment of pre-existing colon polyps and/or the presence of a disease which predisposes an individual towards developing CRC e.g. familial polyposis or Lynch's syndrome and/or prior episode of CRC or colonic adenoma), health status or life style (for example due to high fat diet or heavy alcohol intake).

Since the present inventors have determined that the mean age for diagnosis of CRC in the dataset analysis given below is 73.0 years for males and 74.9 years for females, compositions of the invention may be used to treat, ameliorate and/or prevent CRC in humans aged 50 years or greater, e.g. 55 years or greater such as 60 years or greater, e.g. 60 to 70 years or 65 to 70 or 60 to 75 years.

In other embodiments, the human subject is not constipated and methods of the present invention may both prevent constipation and treat or prevent or ameliorate CRC in such humans.

In other embodiments of the invention, a kit (e.g. package) comprising a plurality of units (for example 5 or more, 10 or more, 20 or more) of compositions of the invention as described supra together with directions for use are provided. For example, the directions for use may state that one, two or three unit(s)/tablet(s) of the invention may be consumed per day either together or in a split dose (e.g. one tablet in the morning, one tablet in the evening). Accordingly therefore a composition in oral unit dosage form (e.g. tablet) for use in a method of treating, ameliorating and/or preventing CRC (and, optionally, preventing constipation) in humans comprises an amount (e.g. effective and sub-laxative amount such as 2.2 grams or thereabout) of a PEG and/or PEG block co-polymer which method comprises administering one, two or three unit(s) of said composition per day. The composition may be used in a method such as described supra.

Compositions of the invention are therefore particularly beneficial for consumption by human subjects on a chronic (for example daily) basis since they have good mouthfeel and structural integrity and provide an amount of PEG useful in the context of colorectal cancer (particularly in conjunction with the methods of the invention) yet avoids the excessive gastrointestinal disturbances (such as loose stools) that may be regarded as unpleasant or generally unacceptable when consuming larger amounts of PEG on such a chronic basis.

It will understood by the reader of this specification, that all embodiments described supra may be construed as to disclose, separately, an embodiment directed to PEG and an embodiment directed to PEG block co-polymer.

It will be apparent to the reader of this specification,that the term “comprising” and grammatical variations thereof, in relation to embodiments of the invention described supra, may instead be “consisting essentially of” or “consisting of”.

The following examples illustrate but do not limit the invention.

### EXAMPLE 1

### Study of Association between Exposure to a Macrogol Laxative and the Risk of Colorectal Cancer

### 1. Study Objectives

To describe the incidence of colorectal cancer on the General Practice Research Database (GPRD) following different types of laxative exposure.

To evaluate any association between exposure to a macrogol laxative and the risk of colorectal cancer.

### 2. Methods

### Data Source and Study Population

### The GPRD

The General Practice Research Database (GPRD) is a computerised database containing anonymised longitudinal data collected within UK primary care. It consists largely of coded data entered onto a computer system by general practitioners (GPs) as part of the clinical management of patients within primary care. Information entered onto the system includes demographic details, symptoms and medical diagnoses, detailed prescription data, hospital referrals and admissions and the results of clinical investigations and tests.

The longitudinal nature of the data combined with large sample sizes and high quality data on consultations, risk factors, diagnoses, prescribing and outcomes means the GPRD has a number of advantages for research. The age and sex distribution of the patient population in the database at any point in time closely matches the Office for National Statistics estimates for the total England and Wales population. In addition, for many co-morbidities, GPRD figures correlate well with those for other UK sources such as cancer statistics and figures reported by the Office for National Statistics. The GPRD is a widely used and validated source of epidemiological data and is a valuable resource for epidemiological research.

### Study Design Considerations

The nature of both the exposure and the outcome of interest for this study meant that the following considerations needed to be taken into account when deciding on the most appropriate study design:

1. Laxative users will differ in terms of their risk of CRC from non-laxative users. Therefore, to diminish the risk of ‘confounding by indication’ any association will need to be evaluated within the population of laxative users;

2. The risk of CRC increases exponentially with age. Therefore it is important that any matching between cases and controls is carried out based on the patients' year of birth and not on wider age bands;

3. CRC has a long latency period and exposure status at the time of diagnosis (classically the way exposure status is measured in epidemiological studies) will not be the relevant exposure status;

4. Changes in bowel movements might be the first presenting symptoms of CRC and therefore care needs to be taken when evaluating any association between laxative use and CRC that the laxatives were not prescribed because of the first presenting symptoms of CRC;

5. Laxative use is often on an ‘as needed’ basis, is intermittent, and people switch between products.

Given the complexities of the association of interest it was concluded that no single study design could provide the complete answer. In order to create a full picture of investigations, it was decided that it would be necessary to carry out a study of CRC incidence rates in different laxative exposure groups combined with a case-control study nested within the cohort study population.

The methods and results of both of these studies will now be discussed in turn.

### A. Descriptive Studies of Laxative Use and CRC Incidence

### Study Period

Movicol® was licensed for use in the UK in 1998 and the study period ran from 1 Jan. 2000 until 31 Mar. 2009. Secular trends in prescribing data presented in this report, however, are given from Jan. 1992 until Dec. 2008: the last full calendar year for which data was available.

### Study Population

The study population consisted of all patients on the GPRD permanently registered at, or transferred out of, a GP practice providing data that the MHRA considered to be up-to-standard for the purposes of research and who had received ≧1 laxative prescription during the time period their medical record was considered to be UTS (up-to-standard). There were no age restrictions.

Identification of Patients with Colorectal Cancer

Colorectal cancer was defined according to the International Classification of Diseases 9th edition and included ICD-9 153-154.1 (inclusive). The GPRD coding system, however, does not use ICD codes, so incident cases of colorectal cancer were identified on the GPRD using a combination of “read codes” and the application of three different algorithms.

### Identification of Laxative Users

Patients were eligible for inclusion in the cohort study from the day after their first recorded laxative prescription onwards. Laxative products were taken as those classified under chapter 1.6.1-1.6.5 of the British National Formulary (BNF). PEG based products at chapter 1.6.4 of the BNF include MOVICOL® which contains approximately 13.125 g of macrogol 3350.

Patients were not included if their only laxative prescription(s) were recorded outside of the time period that their medical record was considered to be up-to-standard (UTS).

### Classification of Laxative Exposure

The laxative exposure of patients was classified into three categories

i. Non-macrogol user only

ii. Macrogol user only or before other laxative use

iii. Macrogol user after other laxative use

Up to the point someone received their first macrogol prescription on the GPRD, they were classified as ‘non-macrogol user only’. From the point of receiving a prescription for a macrogol they were classified as either ‘macrogol user only or before other’ or ‘macrogol user after other’, as appropriate. This was done because, at the time of study, the following uncertainties existed: a) laxatives other than macrogols were not thought to cause any in- or decrease in CRC risk; b) it was unclear whether one dose of macrogol could have an impact on CRC risk, whether there was a threshold effect (a minimum number of dosages was needed to achieve a reduction in CRC risk) or whether there was a dose or duration-response association; and c) whether, if there was a reduction in CRC risk caused by macrogol utilisation, this was because of a reduction in tumour initiation or a reduction in tumour progression.

### B. Case-Control Study

### Study Population

The case-control study was nested within the cohort of laxative users so all cases and controls were recruited from the laxative user cohort identified supra.

Cases and controls were eligible for study inclusion after the date of their first laxative prescription.

### Classification of Laxative Exposure

As with the cohort study, laxative exposure of patients was classified into three categories

i. Non-macrogol use only

ii. Macrogol use only or before other laxative use (‘macrogol first’)

iii. Macrogol use after other laxative use (‘other laxative first’)

Over the course of most of the study period, categories i. and iii. would have reflected common practice in accordance with prescribing guidelines, whereas category ii would have been unusual. This changed in 2006 and it is anticipated to change further following the recent publication of a Cochrane review that concludes macrogols are the laxative of first choice.

### Identification of Colorectal Cancer Cases

Colorectal cancer cases were all patients with CRC newly diagnosed between 1 Jan. 2000 and 31 Mar. 2009 and they were identified using the algorithms and methods described for the cohort study supra. The index date was taken as the earliest date with evidence of a CRC diagnosis and was determined using the same criteria as for the descriptive study of CRC incidence rates. Colorectal cancer cases were required to have ≧6 months of UTS data before the CRC index date to enable information on covariates to be collected.

### Identification of Controls

Six controls were identified for each CRC case in order to achieve optimum statistical power. By definition, controls have to be sampled from the population that gave rise to the cases. Therefore, controls were sampled from the same laxative cohort population and they were required to be disease free (have no diagnosis of colorectal cancer) on and before the index date of the case. In addition, to eliminate confounding by age and sex, they had to have the same year of birth and be of the same sex as the case. Controls were also required to have ≧6 months of UTS data before the index date of the case to enable information on covariates to be collected and they were required to have received ≧1 prescription for a laxative before the CRC index date of the matched case.

### Power Considerations

Based on the assumption macrogols had 10% of the laxative market share and with the selection of 6 controls per case, to demonstrate a 20% reduction in CRC risk the nested case-control study of CRC risk required approximately 2,360 newly diagnosed cases; to demonstrate a 25% reduction in risk 1,480 cases was required. If macrogols had 20% of the market then these numbers became 1,290 and 800 respectively.

### Back Dating of the Colorectal Cancer Index Date

As exposure on the index date was unlikely to be relevant additional sets of controls were selected on the index date minus 6 months, minus 12 months, minus 18, 24, 30, 36, 42, 48, 54 and 60 months and for each of these analysis sets the analyses of exposure and risk factors were carried out based on the ‘new’ index dates. This means that any changes in exposure status or risk factors during the period between the ‘new’ index date and the original index date were ignored on the basis that these were irrelevant because the cancer was already present but had simply not been diagnosed yet.

It was hypothesised that if macrogol laxatives did decrease CRC risk then any association found should become stronger or remain stable with back dating of the index date.

### Dose Response Relationship

For the osmotic laxatives, the total dosage received (millilitres of lactulose and sachets of macrogols) was calculated for all cases and controls. The association between CRC risk and laxative dosage prescribed was evaluated.

### Identification of Data on Potential Risk Factors for Colorectal Cancer

Information was collected, where available, for both cases and controls for each of the covariates listed below. CRC risk is related to diet and information on diet is not recorded within the GPRD. However, information on socioeconomic status (SES) and body mass index (BMI) was included as proxy measures.

Covariates present on the GPRD and reported or suggested to either be risk factors for, or have a protective effect against, colorectal cancer.

### Covariates

Smoking

Alcohol

Body mass index (BMI)

Socioeconomic status (SES)

Inflammatory bowel disease (IBD)

Cancer diagnosis other than colorectal cancer

Diabetes

Cholecystectomy

Prescriptions for:

Low dose aspirin (<300mg)

Non-low dose aspirin (≧300mg)

COX-2 inhibitors

Non-aspirin non-selective NSAIDs

Dantron containing laxatives

Statins

Opioids

Hormone replacement therapy (HRT)

Calcium supplements

5-ASA

### Statistical Analyses

Conditional logistic regression analyses were carried out to evaluate any association between macrogol exposure and colorectal cancer risk. All covariates significant at the level of p<0.20 in the univariate analyses were considered for inclusion in the multivariate models. Covariates remained in the multivariate model if p>0.05 or if they altered the risk estimate by more than 10%. Tests were carried out for interactions between variables and the stability of the models was assessed using the Hosmer-Lemeshow statistic.

### C. Results

### Incidence of Colorectal Cancer on the GPRD.

A total of 14,598 potential incident CRC cases were identified on the GPRD between 1 Jan. 2000 and 31 Mar. 2009, in addition to which there were 255 cases of cancer of the anus that were included for the comparison of CRC incidence on the GPRD with UK figures reported by the cancer registries. Of the total 14,853 colorectal and anus cancer cases identified, 2,402 (16.7%) had a code specifically stating the type of cancer but with no record of supporting evidence for the diagnosis.

### Laxative Drug Utilisation on the GPRD

We identified 872,959 patients on the GPRD who had received a total of 9,866,699 laxative prescriptions between 1 Jan. 1992 and 31 Mar. 2009. A total of 721,513 macrogol laxative prescriptions were identified for 155,609 individuals, of which 1,297 (0.2%) were prescriptions for bowel cleansing preparations (Klean-Prep® or Moviprep®).

The annual number of laxative prescriptions recorded on the GPRD was found to increase over time in line with the increase in the number of patients contributing data to the database. In 2007 a total of 836,391 prescriptions were issued for laxative products. The reason for a slight reduction in the number of prescriptions recorded in 2008 is likely to be the result of the delay in some GP practices uploading their latest data collection to the MHRA. A continuous steady increase was observed in the number of prescriptions for macrogol laxatives following the first marketing authorisation of Movicol®.

Approximately two-thirds of laxative prescriptions on the GPRD were prescribed to females and one-third to males. A slightly larger proportion of macrogol prescriptions were issued to children and adolescents and to adults aged between 30 and 59 compared with all types of laxative prescriptions.

### Case-Control Study

We identified 4,734 eligible incident cases of colorectal cancer. Of the 4,734 CRC cases, 1,592 (33.6%) had only received a prescription for a laxative in the 6 months before the CRC index date.

Of these cases, 49.3% were male and 50.7% were female. CRC is more common in males but the slightly larger proportion of female cases identified is likely to reflect the age and sex distribution of the laxative user cohort combined with the fact that on average, women live longer than men. The mean age at diagnosis was 73.0 years (SD=10.9) and 74.9 years (SD=12.2) for males and females respectively.

These cases were therefore only eligible to be included in the index date analysis set—Table 1.

TABLE 1
Number of cases and matched controls in each
of the backdated analysis sets

| Analysis set | Cases (n) | Controls (n) |
|---|---|---|
| Index date | 4,734 | 28,404 |
| Index date - 6 m | 3,142 | 18,852 |
| Index date - 12 m | 2,722 | 16,332 |
| Index date - 18 m | 2,445 | 14,670 |
| Index date - 24 m | 2,195 | 13,170 |
| Index date - 30 m | 1,982 | 11,892 |
| Index date - 36 m | 1,789 | 10,734 |
| Index date - 42 m | 1,636 | 9,816 |
| Index date - 48 m | 1,481 | 8,886 |
| Index date - 54 m | 1,351 | 8,106 |
| Index date - 60 m | 1,214 | 7,284 |

A potential dose-response relationship was observed for cohort in the “macrogol exposure following other” at 24 months (Table 2) and 36 months (Table 3) before the index date.

TABLE 2
Total Macrogol exposure 24 months before the index date.

| No: of sachets | N (Cases) | Odds Ratio | 95% CI |
|---|---|---|---|
| <20 | 99 (14) | 1.02 | 0.57-1.80 |
| 21-30 | 124 (17) | 0.98 | 0.58-1.66 |
| 31-60 | 154 (22) | 0.97 | 0.62-1.54 |
| 61-180 | 144 (13) | 0.57 | 0.32-1.02 |
| >180 | 140 (11) | 0.50 | 0.27-0.94 |

TABLE 3
Total Macrogol exposure 36 months before the index date

| No. of sachets | N (cases) | Odds Ratio | 95% CI |
|---|---|---|---|
| <20 | 76 (7) | 0.61 | 0.28-1.34 |
| 21-30 | 70 (8) | 0.75 | 0.36-1.59 |
| 31-60 | 94 (11) | 0.80 | 0.42-1.52 |
| 61-180 | 85 (5) | 0.36 | 0.15-0.90 |
| >180 | 82 (6) | 0.45 | 0.20-1.04 |

Since Movicol® contains approximately 13.125 g of macrogol 3350 per sachet, a calculation can be made as to total macrogol exposure over the respective time frames that demonstrate a reduction in risk of CRC. See Tables 4 and 5 below wherein the total macrogol exposure over 24 months and 36 months respectively, together with 12 month, one month, one week and one day equivalent is provided.

TABLE 4
24 month Macrogol exposure (grams).

| | | | One | | |
|---|---|---|---|---|---|
| No: of | | 12 month | month | One week | One day |
| sachets | 24 month | equivalent | equivalent | equivalent | equivalent |
| 61-180 | 800.6-2362.5 | 400.3-1181.3 | 33.3-98.4 | 7.7-22.7 | 1.1-3.2 |
| 180+ | 2362.5+ | 1181.3+ | 98.4+ | 22.7+ | 3.2+ |

TABLE 5
36 month Macrogol exposure (grams)

| | | | One | | |
|---|---|---|---|---|---|
| No: of | | 12 month | month | One week | One day |
| sachets | 36 month | equivalent | equivalent | equivalent | equivalent |
| 61-180 | 800.6-2362.5 | 266.9-787.5 | 22.2-65.6 | 5.1-15.1 | 0.73-2.2 |
| 180+ | 2362.5+ | 787.5+ | 65.6+ | 15.1+ | 2.2+ |

### EXAMPLE 2

### Tablets Containing Different w/w % of Mannitol, Useful in the Invention

The tablets described in Table 6 were prepared by combining the dry ingredients and compressing in a punch and die machine. For tablets 2A to 2C, the machine was a Manesty 16 punch D machine with a standard stainless steel punch and die with flat 22mm diameter and beveled edge with PTFE and vulcalon inserts from I Holland Ltd. For tablets 2D and 2E, the unit formula amounts were compressed on a Manesty D machine at normal manufacturing speed and with a standard stainless steel punch and die with flat 22mm diameter and beveled edge. The properties of the tablets were noted and they are given in Table 6b below.

Tablet 2A had an acceptable taste. However, the tablets were prone to sticking to the tableting machine, and many tablets were capped or laminated, making them unusable. Tablet 2B contained the same flavouring as tablet 2A, but more mannitol (15.3% vs. 9.1% in 2A) and less flavouring (1.5% vs. 5.4%). Tablet 2B had an acceptable taste and there was no evidence of sticking to the tableting machine, or capping or laminating of the tablets. Tablet 2C contains similar amounts of PEG, mannitol and magnesium stearate to tablet 2B, but the flavouring is peppermint. It displays similar characteristics to tablet 2B. Tablet 2D contained no flavouring, and 10% mannitol. It displayed no capping or sticking and only a small amount of chipping. Tablet 2E contained no flavouring, and 40% mannitol. It displayed good manufacturing characteristics. Tablets 2D and 2E had a bland taste as compared with tables 2A to 2C. This is most likely because of the absence of flavouring. The taste was, however, not unpleasant. Tablets 2F to 2H all displayed good manufacturing characteristics and an acceptable taste.

It is seen that a tablet containing from 59.5 to 89.5% w/w PEG (in particular 82.7 or 82.9% w/w PEG) and 10 to 40% /w mannitol (in particular 15.3 or 16.2% w/w mannitol) has better ease of manufacture characteristics than a tablet containing 85.0% PEG and 9.1% mannitol.

TABLE 6a
Composition of Tablets 2A to 2E

| | Tablet 2A | | Tablet 2B | | | Tablet 2C | | | Tablet 2D | | Tablet 2E | |
|---|---|---|---|---|---|---|---|---|---|---|---|---|
| | | 5 kg | | 5 kg | | | 5 kg | | | 5 kg | | 5 kg |
| | Unit | bag | Unit | bag | 255 kg | Unit | bag | 255 kg | Unit | bag | Unit | bag |
| Component | formula | blend | formula | blend | batch | formula | blend | batch | formula | blend | formula | blend |
| PEG av. | 2339 mg | 4.25 kg | 2273.7 mg | 4.144 kg | 211 kg | 2284 mg | 4.155 kg | 212 kg | 2685 mg | 4.48 kg | 1785 mg | 2.98 |
| MW | (85.0%) | | (82.7%) | | | (82.9%) | | | (89.5%) | | (59.5%) | kg |
| 3000-4000 | | | | | | | | | | | | |
| Mannitol | 250 mg | 0.45 kg | 420.75 mg | 0.765 kg | 39 kg | 446 mg | 0.810 kg | 41 kg | 300 mg | 0.50 kg | 1200 mg | 0.75 |
| | (9.1%) | | (15.3%) | | | (16.2%) | | | (10%) | | (40%) | kg |
| Magnesium | 13.75 mg | 0.03 kg | 13.75 mg | 0.025 kg | 1 kg | 13.75 mg | 0.025 kg | 1 kg | 15 mg | 0.03 kg | 15 mg | 0.03 |
| Stearate | (0.5%) | | (0.5%) | | | (0.5%) | | | (0.5%) | | (0.5%) | kg |
| Flavouring | 150 mg | 0.27 kg | 41.8 mg | 0.075 kg | 4 kg | 11 mg | 0.020 kg | 1 kg | — | — | — | — |
| | (5.4%) | | (1.5%) | | | (0.4%) | | | | | | |
| | (raspberry- | | (raspberry- | | | (peppermint) | | | | | | |
| | lemon) | | lemon) | | | | | | | | | |
| Total wt | 2752.75 mg | | 2749.3 mg | | | 2754.75 mg | | | 3000 mg | | 3000 mg | |

| | | |
|---|---|---|
| Tablet 2F | Tablet 2G | Tablet 2H |

| | | 5 kg bag | | 5 kg bag | Unit | |
|---|---|---|---|---|---|---|
| Component | Unit formula | blend | Unit formula | blend | formula | 5 kg bag blend |
| PEG av. MW | 2270 mg | 3.96 Kg | 2045 mg | 3.705 Kg | 1900 mg | 3.46 Kg |
| 3000-4000 | (79%) | | (74%) | | (69%) | |
| Mannitol | 574 mg | 1.0 Kg | 690 mg | 1.25 Kg | 827 mg | 1.5 Kg |
| | (20%) | | (25%) | | (30%) | |
| Magnesium | 14 mg | 0.025 Kg | 14 mg | 0.025 Kg | 14 mg | 0.025 Kg |
| Stearate | (0.49%) | | (0.5%) | | (0.5%) | |
| Flavouring | 11 mg | 0.02 Kg | 11 mg | 0.02 Kg | 11 mg | 0.02 Kg |
| | (Peppermint) | | (peppermint) | | (Peppermint) | |
| | (0.38%) | | (0.4%) | | (0.4%) | |
| Total wt | 2870 mg | 5.0 Kg | 2760 mg | 5.0 Kg | 2756 mg | 5.0 Kg |

TABLE 6b
Properties of Tablets 2A to 2E

| | | | | | |
|---|---|---|---|---|---|
| Ease of | Some capping, | Reliable manufacture with | Reliable manufacture with | Some chipping of | Good control, |
| Manufacture | chipping and | minimal capping, chipping or | minimal capping, chipping or | tablets, but | good appearance |
| | sticking to | sticking at 255 kg scale. Good | sticking at 255 kg scale. No | acceptable. Weight | |
| | punches | weights and hardness | sticking or capping; good | and hardness | |
| | | throughout batch at 5 kg scale. | weight and hardness control; | consistent | |
| | | | good appearance at 5 kg scale. | | |
| Taste | Acceptable taste | Acceptable taste | Acceptable taste | Bland | Bland |
| Hardness | Not measured | 8.95-14.29 kg (measured for | 6.34-9.19 kg (measured for 5 kg | 4.42-7.34 kg | 5.96-10.2 kg |
| | | 5 kg batch) | batch) | | |

Properties of Tablets 2F to 2H

| | | | |
|---|---|---|---|
| Ease of | Appearance of tablet | Appearance of tablet good, no | Appearance of tablet good, no |
| Manufacture | good, no capping | capping | capping |
| Taste | Acceptable taste | Acceptable taste | Acceptable taste |
| Hardness | 6.28-14.4 Kg | 5.12-7.5 Kg | 7.27 Kg-10.15 Kg |

### EXAMPLE 3

### Comparison of Tablets Containing Mannitol and Different w/w % of Magnesium Stearate, Useful in the Invention

The tablets described in Table 7a were prepared. The materials were dispensed and then bag blended. The unit formula amounts were compressed on a Manesty D machine at normal manufacturing speed and with a standard stainless steel punch and die with flat 22 mm diameter and beveled edge and PTFE inserts. The properties of the tablets were noted and they are given in Table 7b below.

It is seen in Tables 7a and 7b that tablets containing PEG and 15% w/w mannitol and 0.2%, 0.5% or 5.0% w/w magnesium stearate have good manufacturing properties and acceptable taste.

TABLE 7a
Composition of Tablets 3A to 3C

| | Tablet 3A | | Tablet 3B | | Tablet 3C | |
|---|---|---|---|---|---|---|
| | Unit | 5 kg bag | | 5 kg bag | | 5 kg bag |
| Component | formula | blend | Unit formula | blend | Unit formula | blend |
| PEG average MW | 2544 mg | 4.24 kg | 2400 mg | 4.0 kg | 2535 mg | 4.23 kg |
| 3000-4000 | (84.8%) | | (80%) | | (84.5%) | |
| Mannitol | 450 mg | 0.75 kg | 450 mg | 0.75 kg | 450 mg | 0.75 kg |
| | (15%) | | (15%) | | (15%) | |
| Magnesium Stearate | 6 mg | 0.01 kg | 150 mg | 0.25 kg | 15 mg | 0.03 kg |
| | (0.2%) | | (5%) | | (0.5%) | |
| Flavouring | — | — | — | — | — | — |
| Total wt | 3000 mg | | 3000 mg | | 3000 mg | |

TABLE 7b
Properties of Tablets 3A to 3C

| | | | |
|---|---|---|---|
| Ease of Manufacture | Tablets good, | Tablets good | Slight capping |
| | no capping | | |
| Taste | Bland taste | Bland taste, | Better taste |
| | | slightly artificial | |
| Hardness | 5.4-11.0 kg | 4.42-7.34 kg | 0.4-6.1 kg |

### EXAMPLE 4

### Comparison of Various Flavours of PEG+Mannitol Tablets, Us the Invention

Tablets analogous to the tablets of Example 2 with a variety of flavourings were prepared by combining the dry ingredients and compressing in a punch and die machine. The machine was a Manesty 16 punch D machine with a standard stainless steel punch and die with flat 22 mm diameter and beveled edge with PTFE and vulcalon inserts from I Holland Ltd.

The tablets were provided to a panel of tasters. They were asked to taste each of the tablets and score them with an emphasis on the flavour, scoring from 1 (unpleasant) to 5 (pleasant). There were 22 tasters and their scores were summed together. It was found that peppermint flavoured tablets (score=65) and lemon-raspberry flavoured tablets (score=87) were preferred over lemon-lime flavoured tablets (score=37) and orange flavoured tablets (score=23).

### EXAMPLE 5

### Comparison of Tablets Containing Various Other Solids, Useful in the Invention

Tablets containing solids other than mannitol were prepared in the same manner as the tablets of Example 2 supra. The composition and properties of these tablets are noted in Table 8a and 8b below.

TABLE 8a
Comparison of Alternative Solids

| | Tablet 5A | Tablet 5B | Tablet 5C |
|---|---|---|---|
| | Unit amount/5 Kg | Unit amount/5 Kg | Unit amount/5 Kg |
| Component | blend | blend | blend |
| PEG av. MW | 2276 mg/4.145 | 2279 mg/4.145 | 2280 mg/ |
| 3000-4000 | Kg (83%) | Kg (83%) | 4.145 Kg |
| Solid | Sorbitol | Lactose/Starch¹ | Xylitol |
| | 445 mg/0.81 Kg | 446 mg/0.81 Kg | 446 mg/0.81 Kg |
| | (16.2%) | (16.2%) | |
| Magnesium | 14 mg/0.025 Kg | 14 mg/0.025 Kg | 14 mg/0.025 Kg |
| Stearate | (0.51%) | (0.51%) | (0.51%) |
| Flavouring | 11 mg/0.02 Kg | 11 mg/0/02 Kg | 11 mg/0.02 Kg |
| | (0.40%) | (0.40%) | (0.40%) |
| Total wt | 2746 mg/5.0 Kg | 2753 mg/5.0 Kg | 2750 mg/5.0 Kg |

¹Lactose/starch compound, StarLac ® (Roquette Pharma, Northants, 1 _ _{—,—} _ _ spray-dried compound consisting of 85% alpha-lactose monohydrate (Ph. Eur./USP-NF) and 15% maize starch (Ph. Eur./USP-NF) dry matter.

TABLE 8b
Properties of Tablets 5A to 5C

| | | | |
|---|---|---|---|
| Ease of | Tablet appearance | Tablet appearance | Tablet appearance |
| Manufacture | good, no capping | good, no capping | good, no capping |
| Taste | Tasted ok but lacked | Good hardness, | Tasted ok, but |
| | enhanced mouthfeel | tasted bland | tablet too soft |
| | of mannitol | | |
| | tablets | | |
| Hardness | 6.97-10.09 Kg | 4.13-9.4 Kg | 4.67-7.99 Kg |

### EXAMPLE 6

### Comparison of Tablets Containing Further Various Solids, Useful in the Invention

Tablets containing solids other than mannitol were prepared in the same manner as the tablets of example 2 supra. The composition and properties of these tablets are noted in Table 9a and 9b below.

TABLE 9a
Comparison of alternative solids

| | Tablet 6A | Tablet 6B | Tablet 6C |
|---|---|---|---|
| | Unit amount/5 Kg | Unit amount/5 Kg | Unit amount/5 Kg |
| Component | blend | blend | blend |
| PEG av. MW | 2279 mg/4.145 | 2279 mg/4.145 | 2263 mg/4.145 |
| 3000-4000 | Kg (82.9%) | Kg (82.9%) | Kg (82.9%) |
| Solid | Lactose | Dextrate¹ | Cellulose² |
| | 445 mg/0.810 Kg | 440 mg/0.800 Kg | 442 mg/0.810 Kg |
| | (16.2%) | (16.0%) | (16.2%) |
| Magnesium | 14 mg/0.025 Kg | 14 mg/0.025 Kg | 13.6 mg/0.025 Kg |
| Stearate | (0.5%) | (0.50%) | (0.5%) |
| Flavouring | 11 mg/0.020 Kg | 11 mg/0.020 Kg | 11 mg/0.020 Kg |
| | (0.4%) | (0.4%) | (0.4%) |
| Total wt | 2750 mg/5.0 Kg | 2746 mg/4.99 Kg | 2730 mg/5.0 Kg |

¹Emdex ®, available from JRS Pharma, Rosenberg, Germany.
²Avicel ®, microcrystalline cellulose, available from FMC biopolymers, Philadelphia, USA.

TABLE 9b
Properties of Tablets 6A to 6C

| | | | |
|---|---|---|---|
| Ease of | Tablet appearance | Tablet appearance | Tablet appearance |
| Manufacture | good, no capping, | good, some capping | good, no capping |
| | hardness good | | |
| Taste | Taste ok, but | Pleasant taste but | Taste unpleasant |
| | very bland | quite soft | |
| Hardness | 5.2-13.6 Kg | 5.4-11.9 Kg | 7.1-13.4 Kg |

1. A method for treating, ameliorating and/or preventing CRC in humans wherein the method comprises administering approximately 800 grams or greater (such as between 800 grams and 2365 grams) of PEG or PEG block co-polymer over a period of 36 consecutive calendar months.

2. A method for treating, ameliorating and/or preventing CRC in humans wherein the method comprises administering approximately 800 grams or greater (such as between 800 grams and 2365 grams) of PEG or PEG block co-polymer over a period of 24 consecutive calendar months.

3. A method for treating, ameliorating and/or preventing CRC in humans wherein the method comprises administering approximately 800 grams or greater (such as between 800 grams and 2365 grams) of PEG or PEG block co-polymer over a period of 18 to 36 consecutive calendar months.

4. A method as claimed in claim 1 wherein the method comprises administering between 0.1 grams and 6.0 grams of PEG or PEG block co-polymer on a daily basis.

5. (canceled)

6. The method of claim 1 for ameliorating CRC in humans by reducing the incidence and/or growth of aberrant crypt foci (ACF).

7. (canceled)

8. The method of claim 1 for treating, ameliorating and/or preventing CRC in humans and for the simultaneous use in preventing constipation and/or maintaining normal gastrointestinal transit time in humans.

9. The method of claim 1 wherein the method comprises administering between 266 and 1181 grams (e.g. between 400.3 to 1181.3 grams or between 266.9 and 787.5 grams or 400.3 and 787.5 grams) of PEG or PEG block co-polymer over a period of 12 consecutive calendar months.

10. The method of claim 1 wherein the method comprises administering between 22 and 98 grams (e.g. between 33.3 to 98.4 grams or between 22.2 and 65.6 grams or 65.6 grams and 98.4 grams) of PEG or PEG block co-polymer over a period of one month.

11. The method of claim 1 wherein the method comprises administering between 5.1 and 22.7 grams (e.g. between 7.7 to 22.7 grams or between 5.1 and 15.1 grams or between 15.1 and 22.7 grams) of PEG or PEG block co-polymer over a period of one week.

12. The method of claim 1 wherein the method comprises administering between 0.73 and 3.22 grams (e.g. between 1.1 to 3.2 grams or between 0.73 and 2.2 grams; or 2.2 to 3.2 grams) of PEG or PEG block co-polymer over a period of one day.

13. The method of claim 1 comprising administering a composition comprising between 0.2 to 6.4 grams of PEG or PEG block co-polymer.

14-16. (canceled)

17. The method of claim 1 comprising administering a composition comprising between 2.0 and 2.5 grams of PEG or PEG block co-polymer.

18. (canceled)

19. The method of claim 13 wherein the composition is administered daily or intermittently .

20. (canceled)

21. The method of claim 1 wherein the PEG has an average molecular weight of between 1000 and 8000 Daltons.

22-23. (canceled)

24. The method of claim 21 wherein the PEG is selected from the group consisting of macrogol 3350, macrogol 4000, macrogol 6000, macrogol 8000.

25. The method of claim 1 wherein the human is predisposed towards developing CRC (e.g. due to familial history and/or medical history such as a prior episode of CRC or colon polyps, Lynch's syndrome, familial polyposis and/or health status and/or lifestyle).

26. The method of claim 1 wherein the human is aged 50 years or greater e.g. 55 years or greater such as 60 years or greater e.g. 60 to 75 years or 65 to 75 years.

27. The method of claim 1 comprising administering a composition in oral dosage form.

28. (canceled)

29. A method as claimed in claim 1 for treating, ameliorating and/or preventing CRC in humans which comprises administering a composition comprising:
(a) 50-90% w/w polyethylene glycol (PEG), having an average molecular weight within the range 2,000 to 10,000 Da; and
(b) 10-40% w/w of a solid;
(c) optionally further excipients such as flavourings, sweeteners and lubricants

30. The method according to claim 29 wherein the composition is a solid composition for oral administration as a solid.

31-36. (canceled)

37. The method of claim 29 comprising administering a composition comprising:
(a) 70-90% w/w polyethylene glycol (PEG) having an average molecular weight within the range 2,000 to 10,000 Da;
(b) 10-20% w/w of the solid such as mannitol;
(c) 0-2.0% w/w lubricant; and
(d) 0-2.0% w/w flavouring.

38-42. (canceled)

43. The method of claim 29 comprising administering a composition that is substantially free from electrolytes (e.g. sodium chloride, potassium chloride, bicarbonates such as sodium bicarbonate, sulphates such as sodium sulphate or phosphates).

44. (canceled)

45. The method as claimed in claim 29 comprising administering a composition that has a mass of 2.0 to 3.5 g and comprises:
(a) 1.00-3.15 g polyethylene glycol (PEG) having an average molecular weight within the range 2,000 to 10,000;
(b) 0.20-1.40 g mannitol.

46-51. (canceled)
